(19) United States
(12) Patent Application Publication
(10) Pub. No.: US 2026/0209141 A1
(43) Pub. Date: Jul. 23, 2026

(54) HYDROCARBON PRODUCTION SYSTEM AND HYDROCARBON PRODUCTION METHOD

(71) Applicant: MITSUBISHI HEAVY INDUSTRIES, LTD., Tokyo (JP)

(72) Inventors: Koichiro YOSHITOKU, Tokyo (JP); Atsuhiro YUKUMOTO, Tokyo (JP); Yukio TANAKA, Tokyo (JP); Yoshio SEIKI, Tokyo (JP)

(73) Assignee: MITSUBISHI HEAVY INDUSTRIES, LTD., Tokyo (JP)

(21) Appl. No.: 19/140,255

(22) PCT Filed: Oct. 25, 2023
(86) PCT No.: PCT/JP2023/038486

§ 371 Date: Jun. 17, 2025

(30) Foreign Application Priority Data
Dec. 20, 2022 (JP) 2022-203137

(51) Int. Cl. C07C 2/82; B01D 53/22; B01J 19/00; B01J 19/24; C07C 1/12; C25B 1/04; C25B 15/08; F25J 3/04

(52) U.S. Cl. CPC C07C 2/82; B01D 53/229; B01J 19/0013; B01J 19/245; C07C 1/12; C25B 1/04; C25B 15/081; F25J 3/04636; B01D 2053/221; B01D 2256/10; B01D 2256/12; B01D 2257/504; B01J 2219/00144; F25J 2215/56

(57) ABSTRACT

A hydrocarbon production system that generates a hydrocarbon having two or more carbon atoms from methane by an oxidative coupling reaction of methane is a hydrocarbon production system including: an oxidative coupling reaction device configured to perform an oxidative coupling reaction between methane and oxygen; a raw gas separation device configured to separate an inert component from a raw gas containing the inert component; a carbon dioxide separation device configured to separate carbon dioxide contained in a generated gas generated in the oxidative coupling reaction device; and a methanation device configured to perform a methanation reaction between hydrogen and carbon dioxide.

### TECHNICAL FIELD

The present disclosure relates to a hydrocarbon production system and a hydrocarbon production method.

Priority is claimed on Japanese Patent Application No. 2022-203137, filed Dec. 20, 2022, the content of which is incorporated herein by reference.

### BACKGROUND ART

The technology for producing hydrocarbons such as olefins by an oxidative coupling reaction (“oxidative coupling of methane”, hereinafter, also referred to as an “OCM reaction”) using a gas containing methane such as natural gas is known.

For example, Patent Document 1 proposes a hydrocarbon production system that supplies a purge gas to a gas turbine system including a device that converts a gas containing carbon dioxide, carbon monoxide, and hydrogen into methane at a rear stage of an oxidative coupling reaction device to recycle the converted methane, and a high-pressure steam generating device (HRSG). According to the invention of Patent Document 1, heat or power is used in the hydrocarbon production system from the steam system connected to the HRSG by supplying the purge gas.

### CITATION LIST

### Patent Document

Patent Document 1: United States Patent Application, Publication No. 2016/0272556

### SUMMARY OF INVENTION

### Technical Problem

However, in the invention of Patent Document 1, the purge gas is used as fuel for a gas turbine, the fuel is combusted in the gas turbine, and carbon dioxide is emitted. In addition, a part of carbon dioxide, recovered from a carbon dioxide separation device, of a generated gas generated by the oxidative coupling reaction is used for methanation, but the surplus thereof has no use application and is emitted into the atmosphere. On the other hand, in recent years, there has been an increasing movement towards carbon neutrality in each industry.

The present disclosure has been made to achieve the above-described problems, and an object of the present disclosure is to provide a hydrocarbon production system and a hydrocarbon production method capable of reducing the emission amount of carbon dioxide in a case where a hydrocarbon is produced by an OCM reaction.

### Solution to Problem

In order to solve the above-described problems, a hydrocarbon production system according to the present disclosure is a hydrocarbon production system that generates a hydrocarbon having two or more carbon atoms from methane by an oxidative coupling reaction of methane, the hydrocarbon production system including: an oxidative coupling reaction device configured to perform an oxidative coupling reaction between methane and oxygen; a raw gas separation device configured to separate an inert component from a raw gas containing the inert component; a carbon dioxide separation device configured to separate carbon dioxide contained in a generated gas generated in the oxidative coupling reaction device; and a methanation device configured to perform a methanation reaction between hydrogen and carbon dioxide.

A hydrocarbon production method according to the present disclosure is a hydrocarbon production method for generating a hydrocarbon having two or more carbon atoms from methane by an oxidative coupling reaction of methane, the hydrocarbon production method including: a step of generating a hydrocarbon having two or more carbon atoms by an oxidative coupling reaction of methane; a step of separating an inert component from a raw gas containing the inert component; a step of separating carbon dioxide contained in a generated gas generated by the oxidative coupling reaction; and a step of generating methane from hydrogen and carbon dioxide by a methanation reaction.

### Advantageous Effects of Invention

According to the hydrocarbon production system and the hydrocarbon production method of the present disclosure, the emission amount of carbon dioxide can be reduced in a case where a hydrocarbon is produced by the OCM reaction.

### BRIEF DESCRIPTION OF DRAWINGS

FIG. 1 A schematic view showing a configuration of a hydrocarbon production system according to a first embodiment of the present disclosure.

FIG. 2 A flowchart showing a hydrocarbon production method according to the first embodiment of the present disclosure.

FIG. 3 A schematic diagram of a simplified model of an OCM plant in a case where the effect of nitrogen concentration in high-purity oxygen gas is simulated.

FIG. 4 A schematic view showing a configuration of a hydrocarbon production system according to a second embodiment of the present disclosure.

FIG. 5 A schematic view showing a configuration of a hydrocarbon production system according to a third embodiment of the present disclosure.

### DESCRIPTION OF EMBODIMENTS

### First Embodiment

### <<Hydrocarbon Production System>>

A hydrocarbon production system of the present disclosure includes an oxidative coupling reaction device, a raw gas separation device, a carbon dioxide separation device, and a methanation device.

Hereinafter, a first embodiment of the present disclosure will be described in detail with reference to the drawings.

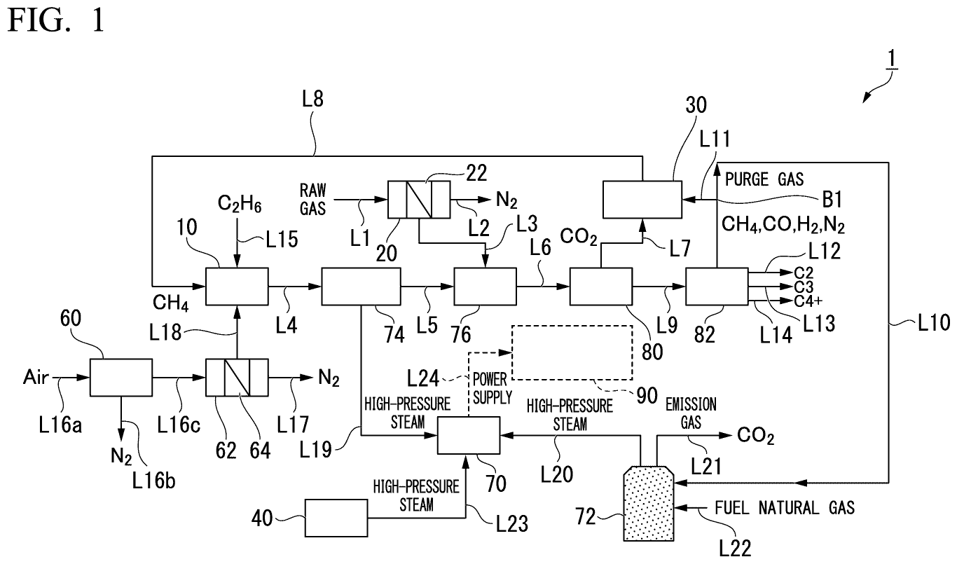

As shown in FIG. 1, the hydrocarbon production system 1 according to the present embodiment includes an oxidative coupling reaction device 10, a raw gas separation device 20, a methanation device 30, and a carbon dioxide separation device 80.

A heat recovery device 74 is connected to the oxidative coupling reaction device 10 by a pipe L4. The booster device 76 is connected to the heat recovery device 74 by a pipe L5. The carbon dioxide separation device 80 is connected to the booster device 76 by a pipe L6. The methanation device 30 and the carbon dioxide separation device 80 are connected to each other by a pipe L7. The oxidative coupling reaction device 10 and the methanation device 30 are connected to each other by a pipe L8. A pipe L15 is connected to the oxidative coupling reaction device 10. The raw gas separation device 20 and the booster device 76 are connected to each other by a pipe L3. A pipe L1 and a pipe L2 are connected to the raw gas separation device 20. A hydrocarbon separation device 82 is connected to the carbon dioxide separation device 80 by a pipe L9.

A steam system 70 is connected to the heat recovery device 74 by a pipe L19. An auxiliary boiler 72 is connected to the steam system 70 by a pipe L20. The auxiliary 10 boiler 72 and the hydrocarbon separation device 82 are connected to each other by a pipe L10. A pipe L21 and a pipe L22 are connected to the auxiliary boiler 72. A pipe L12, a pipe L13, and a pipe L14 are connected to the hydrocarbon separation device 82. A branch B1 of the pipe L10 and the methanation device 30 are connected to each other by a pipe L11.

A cryogenic separation device 60 and an oxygen separation device 62 are connected to each other by a pipe L16c. A pipe L16a and a pipe L16b are connected to the cryogenic separation device 60. The oxygen separation device 62 is connected to the oxidative coupling reaction device 10 by a pipe L18. A pipe L17 is connected to the oxygen separation device 62.

A solar steam system 40 is connected to the steam system 70 by a pipe L23. A power supply line L24 is connected to the steam system 70. The steam system 70 and utility devices 90 are connected to each other by the power supply line L24.

Hereinafter, each configuration of the hydrocarbon production system 1 will be described in detail.

### <Oxidative Coupling Reaction Device>

The oxidative coupling reaction device 10 is a device that performs an oxidative coupling reaction for generating a hydrocarbon having two or more carbon atoms from methane and oxygen.

Examples of the oxidative coupling reaction device 10 include a reactor equipped with a catalyst. In the oxidative coupling reaction device 10, in addition to the oxidative coupling reaction that generates a hydrocarbon having two or more carbon atoms and water, side reactions such as a combustion reaction and a steam reforming reaction occur. Therefore, carbon dioxide, carbon monoxide, and hydrogen are simultaneously generated.

### <Heat Recovery Device>

The heat recovery device 74 is a device that recovers the reaction heat generated in the oxidative coupling reaction device 10 and cools the generated gas.

Examples of the heat recovery device 74 include a waste heat boiler and a heat exchanger. As the heat recovery device 74, the waste heat boiler is preferable since the recovered heat can be supplied to the steam system 70 as high-pressure steam.

### <Booster Device>

The booster device 76 is a device that pressurizes the cooled generated gas and supplies the pressurized generated gas to the carbon dioxide separation device 80.

Examples of the booster device 76 include a pressurizing device such as a compressor.

### <Carbon Dioxide Separation Device>

The carbon dioxide separation device 80 is a device that separates carbon dioxide contained in the generated gas and supplies the separated carbon dioxide to the methanation device 30. The surplus carbon dioxide in the methanation reaction is released into the atmosphere.

Examples of the carbon dioxide separation device 80 include a device equipped with a carbon dioxide absorbing liquid. Examples of the carbon dioxide absorbing liquid include an amine-based absorbing liquid.

### <Methanation Device>

The methanation device 30 is a device that performs a methanation reaction that generates methane from hydrogen, carbon dioxide, and carbon monoxide.

Examples of the methanation device 30 include a reactor equipped with a catalyst.

### <Hydrocarbon Separation Device>

The hydrocarbon separation device 82 is a device that separates and purifies the hydrocarbon contained in the generated gas.

Examples of the hydrocarbon separation device 82 include a column such as a distillation column.

### <Raw Gas Separation Device>

The raw gas separation device 20 is a device that separates an inert component from a raw gas containing natural gas and the inert components. Examples of the inert component include nitrogen, argon, and helium. The inert component contains the largest amount of nitrogen.

Examples of the raw gas separation device 20 include a container equipped with an inert component separation membrane 22. Examples of the inert component separation membrane 22 include a hollow fiber membrane formed of a polymer material and a ceramic membrane that can be applied even at high temperatures and high pressures.

The raw gas separation device may have other separation functions instead of the inert component separation membrane. Examples of the other separation functions include a separation function using an adsorbent. Examples of the adsorbent include adsorbents such as a molecular sieve and zeolite.

### <Cryogenic Separation Device>

The cryogenic separation device 60 is a device that removes an inert component from air and separates oxygen from air.

Examples of the cryogenic separation device 60 include a device such as a cryogenic separation distillation column that can perform rough separation of inert components such as nitrogen.

### <Oxygen Separation Device>

The oxygen separation device 62 is a device that further separates high-purity oxygen (high-purity oxygen gas) from the oxygen separated by the cryogenic separation device 60. The oxygen separation device 62 has an oxygen separation membrane 64 that separates high-purity oxygen gas.

Examples of the oxygen separation device 62 include a high-level purification device equipped with the oxygen separation membrane 64. Examples of the oxygen separation membrane 64 include a hollow fiber membrane formed of a polymer material and a thin membrane having a porous body formed of ceramics.

### <Steam System>

The steam system 70 is a device that converts high-pressure steam into power or supplies heat.

Examples of the steam system 70 include a system composed of a steam turbine for supplying power to a chemical plant and a steam header for supplying steam to the steam turbine, a heat exchanger, and the like.

### <Auxiliary Boiler>

The auxiliary boiler 72 is a device that combusts gas to generate high-pressure steam through the heat produced by the combustion.

Examples of the auxiliary boiler 72 include a known gas boiler. As the fuel for the auxiliary boiler 72, for example, in addition to the purge gas emitted from the hydrocarbon separation device 82 for the purpose of managing the concentration of an inert component in a recycling gas to be described below, fuel natural gas and the like can be used.

### <Utility Device>

The utility devices 90 are various devices in the hydrocarbon production system 1 that require heat or power.

Examples of the utility devices 90 include the cryogenic separation device 60, the booster device 76, and the hydrocarbon separation device 82.

### <Solar Steam System>

The solar steam system 40 is a device that generates high-pressure steam by using thermal energy from sunlight.

Examples of the solar steam system 40 include a system equipped with a storage tank that stores molten salt, an evaporator that causes the heat of the molten salt in the storage tank to be absorbed in water to generate steam, a mirror (for example, a heliostat), and a concentrator that collects sunlight reflected by the mirror and heats the molten salt circulated from the storage tank.

The pipes L1 to L23 are not particularly limited, and examples thereof include a metal pipe, a resin pipe, and a pipe using a composite material in which carbon fiber is mixed into resin.

Examples of the power supply line L24 include equipment such as a coaxial or gear-equipped compressor turbine. The power supply line L24 may function as a heat supply line. In that case, examples of the power supply line L24 include a pipe such as a steam pipe.

### <<Hydrocarbon Production Method>>

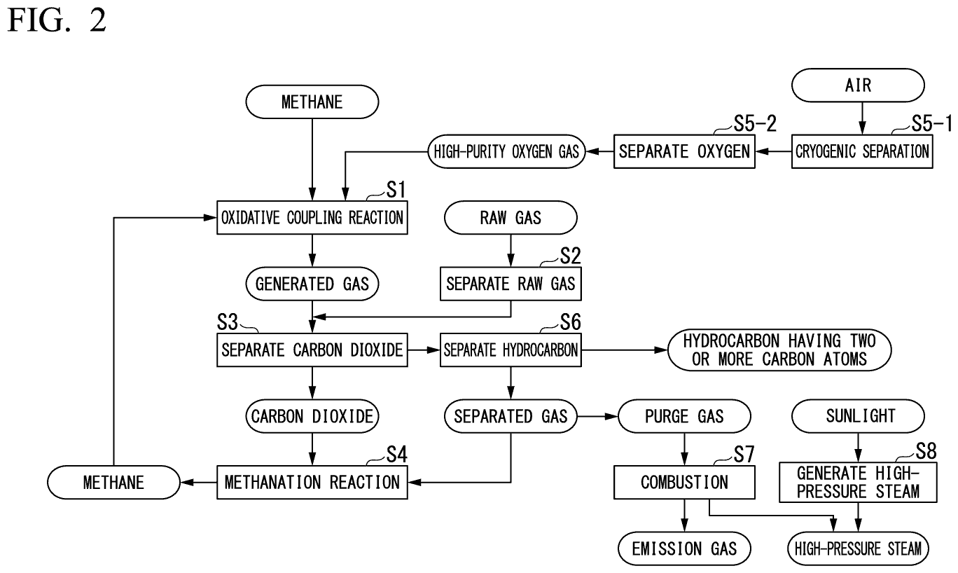

As shown in FIG. 2, the hydrocarbon production method of the present disclosure includes a step (S1) of generating a hydrocarbon having two or more carbon atoms from methane by an oxidative coupling reaction, a step (S2) of separating an inert component from a raw gas containing methane and the inert component, a step (S3) of separating carbon dioxide contained in the generated gas generated by the oxidative coupling reaction, and a step (S4) of generating methane from hydrogen and carbon dioxide by a methanation reaction.

Hereinafter, the hydrocarbon production method of the present embodiment will be described with reference to the hydrocarbon production system 1 in FIG. 1. Reference numerals S1 to S8 mean reference numerals in FIG. 2.

First, a raw gas containing methane and an inert component is supplied from the pipe L1 to the raw gas separation device 20.

Examples of the raw gas include natural gas, liquefied natural gas, and methane fermentation gas.

The raw gas supplied to the raw gas separation device 20 has its inert component separated by the inert component separation membrane 22, and a hydrocarbon gas and carbon dioxide (hereinafter, also referred to as a “first gas”) are obtained (S2). The separated inert component (main component: nitrogen) flows through the pipe L2 and is emitted to the outside. The first gas flows through the pipe L3 and is supplied to the booster device 76.

Since the inert component is removed from the first gas, the amount of gas (hereinafter, also referred to as “purge gas”) emitted from the subsequent hydrocarbon separation device 82 can be reduced. Therefore, the emission amount of carbon dioxide due to the combustion of the purge gas can be reduced.

Here, an example in which the inert component separation membrane 22 is used as a method for obtaining the first gas has been described, but the present disclosure is not limited thereto, and methods using an absorbing liquid, an adsorbent, a distillation column, or the like may be used.

The first gas supplied to the booster device 76 is mixed with the gas (hereinafter, also referred to as “generated gas”) generated in the oxidative coupling reaction device 10, and is pressurized in the booster device 76 as a mixed gas.

The pressure at which the mixed gas is pressurized is preferably, for example, 2 to 4 MPaG in terms of gauge pressure. In a case where the pressure at which the mixed gas is pressurized is equal to or higher than the above-described lower limit value, the separation efficiency in the subsequent carbon dioxide separation device 80 or hydrocarbon separation device 82 can be further increased, and the hydrocarbon production system 1 can be made more compact. In a case where the pressure at which the mixed gas is pressurized is equal to or lower than the above-described upper limit value, the energy required for the pressurization can be saved.

The pressurized mixed gas flows through the pipe L6 and is supplied to the carbon dioxide separation device 80. The mixed gas supplied to the carbon dioxide separation device 80 is separated into carbon dioxide and another gas (hereinafter, also referred to as a “second gas”) by an absorption method or an adsorption method (S3). Examples of the absorption method include a method using an absorbing liquid that selectively absorbs carbon dioxide. Examples of the absorbing liquid include an amine-based absorbing liquid. Examples of the adsorption method include a method using an adsorbent, which selectively adsorbs carbon dioxide, or a separation membrane. Examples of the adsorbent include adsorbents such as a molecular sieve and zeolite. Examples of the separation membrane include a ceramic membrane that can withstand high pressure. The separated carbon dioxide flows through the pipe L7 and is supplied to the methanation device 30, and the surplus carbon dioxide is emitted to the outside. The separated second gas flows through the pipe L9 and is supplied to the hydrocarbon separation device 82.

The second gas supplied to the hydrocarbon separation device 82 is separated into a target hydrocarbon having two or more carbon atoms (for example, ethylene, propylene, butylene, or the like) by a distillation method or the like (S6). The hydrocarbon having 2 carbon atoms is recovered from the pipe L12 and further separated into ethylene and ethane by a distillation column. A hydrocarbon having 3 carbon atoms is recovered from the pipe L13. A hydrocarbon having 4 or more carbon atoms is recovered from the pipe L14. In this case, a saturated hydrocarbon such as ethane is supplied from the pipe L15 to the oxidative coupling reaction device 10 and recycled.

The remaining gas (hereinafter, also referred to as “separated gas”) from which the target hydrocarbon is separated by the hydrocarbon separation device 82 includes unreacted methane, carbon monoxide, hydrogen, nitrogen, and the like. The separated gas flows through the pipe L10, the branch B1, and the pipe L11, is supplied to the methanation device 30, and is recycled. On the other hand, the remaining separated gas flows through the pipe L10 as a purge gas and is supplied to the auxiliary boiler 72.

In the methanation device 30, methane is generated by a reaction (methanation reaction) of the following Formula (1) between the carbon dioxide separated by the carbon dioxide separation device 80 and the hydrogen contained in the separated gas (S4). In addition, in the methanation device 30, the carbon monoxide contained in the separated gas also contributes to the reaction, and methane is generated by the reaction of Formula (2). In the methanation device 30, only the reaction of Formula (1) may proceed, or both reactions of Formulae (1) and (2) may proceed. That is, the raw material of the methanation reaction may include carbon monoxide.

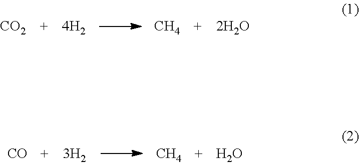

The methane contained in the separated gas flows through the pipe L8 as a recycling gas together with the methane generated by the methanation reaction and is supplied to the oxidative coupling reaction device 10.

In the hydrocarbon production system 1, the raw gas is recycled. In a case where no measure is performed on the inert component that is not consumed in the oxidative coupling reaction, the inert component accumulates in the recycling gas by recycling, the concentration of the inert component in the recycling gas increases, and the reaction efficiency of the oxidative coupling reaction decreases. In order to manage the concentration of the inert component in the recycling gas, a part of the recycling gas (the remaining gas from which the target hydrocarbon separated by the hydrocarbon separation device 82 is separated) is extracted as a purge gas and is supplied to the auxiliary boiler 72 as fuel through the pipe L10.

Meanwhile, air is supplied from the pipe L16a to the cryogenic separation device 60. The air supplied to the cryogenic separation device 60 is roughly separated into an inert component such as nitrogen and a gas (hereinafter, also referred to as a “high-concentration oxygen gas”) containing a large amount of oxygen by cryogenic separation (S5-1). The inert component such as nitrogen flows through the pipe L16b and is emitted to the outside. The high-concentration oxygen gas flows through the pipe L16c and is supplied to the oxygen separation device 62. The high-concentration oxygen gas supplied to the oxygen separation device 62 is highly purified as a high-purity oxygen gas in which a very small amount of an inert component such as nitrogen is separated by the oxygen separation membrane 64 and the oxygen concentration is higher than the oxygen concentration of the high-concentration oxygen gas (S5-2). The separated inert component such as nitrogen flows through the pipe L17 and is emitted to the outside. The high-purity oxygen gas flows through the pipe L18 and is supplied to the oxidative coupling reaction device 10.

Here, an example in which the oxygen separation membrane 64 is used as a method for generating a high-purity oxygen gas has been described, but the present disclosure is not limited thereto, and a method using an absorbing liquid, an adsorbent, a distillation column, or the like may be used.

By removing the inert component such as nitrogen from the air by the cryogenic separation device 60, the amount of the inert component such as nitrogen in the high-purity oxygen gas supplied to the oxidative coupling reaction device 10 can be reduced. Therefore, the amount of the inert component such as nitrogen in the separated gas and the purge gas emitted from the subsequent hydrocarbon separation device 82 can be reduced. As a result, the emission amount of the carbon dioxide due to the combustion of the purge gas can be reduced.

The supply of the air to the cryogenic separation device 60 and the supply of the raw gas to the raw gas separation device 20 are continuously performed.

In the oxidative coupling reaction device 10, a hydrocarbon (mainly ethylene) having two or more carbon atoms is generated (S1) by the reaction (oxidative coupling reaction of methane, OCM reaction) of Formula (3) from methane contained in the recycling gas and oxygen contained in the high-purity oxygen gas supplied from the oxygen separation device 62.

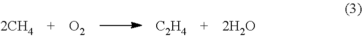

In the oxidative coupling reaction device 10, in addition to the oxidative coupling reaction that generates a hydrocarbon having two or more carbon atoms and water, side reactions such as a combustion reaction and a steam reforming reaction of the following Formulae (4) to (6) occur. Therefore, carbon dioxide, carbon monoxide, and hydrogen are simultaneously generated. In the side reactions, not only methane but also a hydrocarbon having two or more carbon atoms can also be used as a raw material for the side reactions.

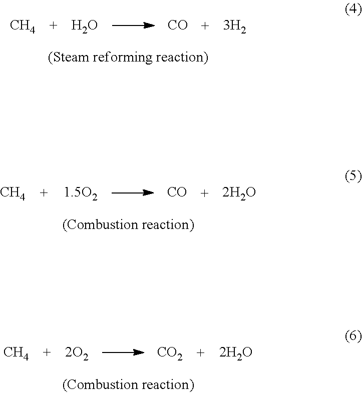

The gas (generated gas) generated by the OCM reaction flows through the pipe L4 and is supplied to the heat recovery device 74.

The temperature in the OCM reaction is, for example, preferably 400° C. to 1000° C. In a case where the temperature in the OCM reaction is equal to or higher than the above-described lower limit value, the generation of a hydrocarbon having two or more carbon atoms is further promoted due to an increase in reaction rate. In a case where the temperature in the OCM reaction is equal to or lower than the above-described upper limit value, side reactions such as a steam reforming reaction and a combustion reaction can be suppressed, and a decrease in the coupling selectivity can be suppressed.

In the present specification, the methane conversion rate is defined by the following Expression (7).

$$Methane conversion rate ( \% ) = ( methane flow rate at reactor inlet - methane flow rate at reactor outlet ) / methane flow rate at reactor inlet \times 100 \qquad ( 7 )$$

In the present specification, the coupling selectivity is defined by the following Expression (8).

$$Coupling selectivity ( \% ) = ( methane conversion C 2 + compound flow rate ) / ( methane flow rate at reactor inlet - methane flow rate at reactor outlet ) \times 100 \qquad ( 8 )$$

The unit of each flow rate in the above expression is a volume-based flow rate (for example, Ncc/min) in a reference state (normal), and the “methane conversion C2+compound flow rate” in the definition of the coupling selectivity means a flow rate converted into methane by multiplying the flow rate of each compound of a hydrocarbon having two or more carbon atoms, such as ethane, ethylene, propane, and propylene, by the number of carbon atoms of each compound.

The pressure in the OCM reaction is preferably, for example, 0.4 to 2 MPaG in terms of gauge pressure. In a case where the pressure in the OCM reaction is equal to or greater than the above-described lower limit value, the gas volume is reduced due to pressurization. Therefore, the oxidative coupling reaction device 10 can be made compact. In a case where the pressure in the OCM reaction is equal to or lower than the above-described upper limit value, a decrease in the coupling selectivity can be suppressed, and a decrease in the yield of a hydrocarbon having two or more carbon atoms can be further suppressed.

The molar ratio of oxygen to methane in the OCM reaction is, for example, preferably 1:4 to 1:20. In a case where the molar ratio of oxygen to methane in the OCM reaction is 1:4 or more, it is easy to prevent the side reactions (combustion reaction and the like) of the oxidative coupling reaction from excessively proceeding, to prevent a decrease in the coupling selectivity, and to prevent a decrease in the yield of a hydrocarbon having two or more carbon atoms. In a case where the molar ratio of oxygen to methane in the OCM reaction is 1:20 or less, it is easy to prevent the oxidative coupling reaction between being limited, to prevent a decrease in the methane conversion rate, and to prevent a decrease in the yield of the hydrocarbon having two or more carbon atoms.

The molar ratio of oxygen to methane in the OCM reaction can be adjusted by the flow rate of the high-purity oxygen gas. In a case where all the oxygen as a raw material is consumed in the reactor of the oxidative coupling reaction device 10, the oxidative coupling reaction does not proceed any further. Therefore, the reaction amount can be controlled by adjusting the oxygen supply amount at the reactor inlet.

In addition, by using a plurality of reactors and placing a cooler between the reactors, the gas temperature increased by an exothermic reaction can be adjusted to an appropriate temperature. In that case, oxygen as a raw material may be supplied between the reactors.

The ethane (C₂H₆) supplied from the pipe L15 to the oxidative coupling reaction device 10 can be supplied to a reactor after a reaction (oxidative coupling reaction) from methane and oxygen is completed, and ethylene and hydrogen can be obtained by thermal decomposition of ethane. As a result, the yield of ethylene, which is a target product, can be increased. The ethane used as a raw material may be provided by recycling the ethane obtained by the hydrocarbon separation device 82 or may be introduced from the outside of the hydrocarbon production system 1. In addition, since the thermal decomposition of ethane is an endothermic reaction, a high-temperature generated gas obtained by the oxidative coupling reaction can be cooled. In that case, a heater may not be used for the thermal decomposition of ethane.

Here, an example in which the influence of the nitrogen concentration in the high-purity oxygen gas on the flow rate (content) of methane in the purge gas in the OCM reaction is simulated will be described.

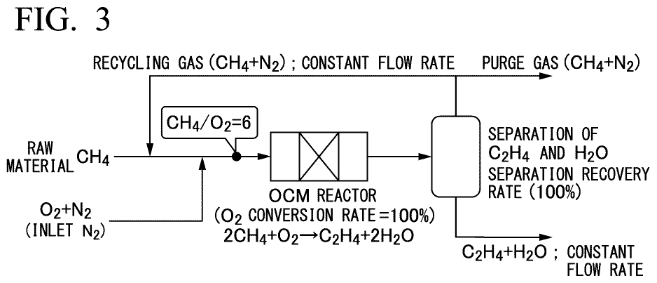

In the simulation, mass balance analysis was performed with a model (see FIG. 3) in which an OCM plant was simplified. The total amount of the recycling gas (methane+N₂ gas) and the reaction rate (methane conversion rate, amount of ethylene (C₂H₄) generated) of the oxidative coupling reaction were assumed, and the oxygen supply amount (CH₄/O₂ ratio) required for the oxidative coupling reaction was calculated from the assumed values. Three types of simulations were carried out in which the nitrogen concentration (inlet N₂) in the high-purity oxygen gas was set to 0.0 mol % in Example 1, 0.5 mol % in Example 2, and 1.0 mol % in Example 3, and the amounts of CH₄ and N₂ in the purge gas were calculated. The results are shown in Table 1.

TABLE 1

| | | Example 1 | Example 2 | Example 3 |
|---|---|---|---|---|
| Inlet N₂ | mol % | 0.0 | 0.5 | 1.0 |
| Reactor inlet CH₄/O₂ | mol/mol | 6.0 | 6.0 | 6.0 |
| Total recycled flow rate | kmol/h | 150 | 150 | 150 |
| CH₄ conversion rate | % | 33.33 | 33.33 | 33.33 |
| Generated C₂H₄ | kmol/h | 50 | 50 | 50 |
| CH₄ in purge gas | mol % | 100.00 | 99.51 | 99.03 |
| | kmol/h | 50.00 | 50.74 | 51.46 |
| N₂ in purge gas | mol % | 0.00 | 0.49 | 0.97 |
| | kmol/h | 0.00 | 0.25 | 0.51 |
| CH₄ in purge gas/ | — | 1.00 | 1.01 | 1.03 |
| Generated C₂H₄ | | | | |
| N₂ in purge gas/ | — | 0.000 | 0.005 | 0.010 |
| Generated C₂H₄ | | | | |

As shown in Table 1, the flow rate of methane in the purge gas was 50.00 kmol/h in Example 1, 50.74 kmol/h in Example 2, and 51.46 kmol/h in Example 3. In this way, it was confirmed that the flow rate of methane and the flow rate of nitrogen in the purge gas can be reduced by reducing the concentration of the inlet N₂. This indicates that the flow rates of the separated gas and the purge gas can be reduced by reducing the nitrogen concentration in the high-purity oxygen gas. The results of the simulation in Table 1 show that a purge gas fuel-derived emission gas generated by combustion in the auxiliary boiler 72 can be reduced, and the emission amount of carbon dioxide can be reduced.

The OCM reaction is an exothermic reaction. Therefore, the generated gas supplied to the heat recovery device 74 has a high temperature (for example, 900° C.). In the heat recovery device 74, heat is exchanged between the high-temperature generated gas and the boiler supply water, and the high-pressure steam is allowed to flow through the pipe L19 and is supplied to the steam system 70. The cooled generated gas flows through the pipe L5 and is supplied to the booster device 76.

The generated gas supplied to the booster device 76 is mixed with the first gas separated by the raw gas separation device 20, and is pressurized in the booster device 76 as a mixed gas.

In the auxiliary boiler 72, the purge gas and natural gas (fuel natural gas) serving as fuel are combusted to generate high-pressure steam and emission gas (S7). The emission gas is emitted to the outside after flowing through a pipe L21 after harmful substances such as nitrogen oxide are removed by an emission gas treatment device (not shown). The high-pressure steam flows through the pipe L20 and is supplied to the steam system 70.

In the present embodiment, since the amount of purge gas is reduced, the supply amount of the fuel natural gas supplied from the pipe L22 is increased to maintain the amount of the high-pressure steam generated by the auxiliary boiler 72.

In the solar steam system 40, high-pressure steam is generated by using thermal energy from sunlight (S8). The solar steam system 40 can generate high-pressure steam without emitting carbon dioxide. Therefore, in a case where the amount of purge gas supplied to the auxiliary boiler 72 is reduced, the emission amount of carbon dioxide emitted from the plant can be reduced by using the solar steam system 40 without increasing the supply amount of the fuel natural gas to maintain the amount of high-pressure steam.

In addition, by enhancing the facilities of the solar steam system 40 to further increase the amount of steam generated, the emission amount of carbon dioxide emitted from the plant can be further reduced by reducing the supply amount of the fuel natural gas supplied to the auxiliary boiler 72.

The high-pressure steam generated in the solar steam system 40 is supplied to the steam system 70 via the pipe L23.

The hydrocarbon production system 1 is a system that produces a hydrocarbon having two or more carbon atoms, such as ethylene, in a constant amount. Therefore, it is preferable that the amount of high-pressure steam supplied to the steam system 70 that generates power also remains constant. Since the solar steam system 40 is affected by the weather, the amount of high-pressure steam generated may change over time.

In the hydrocarbon production system described in Patent Document 1, since both the waste heat boiler that recovers reaction heat to generate steam and the high-pressure steam generating device (HRSG) of the gas turbine use waste heat, the amount of steam generated cannot be adjusted. Therefore, there is no advantage to combining this system with the solar steam system 40 that is affected by the weather.

On the other hand, in the hydrocarbon production system 1 using the auxiliary boiler 72, the shortfall obtained by subtracting the amount of high-pressure steam from the solar steam system 40 from the amount of high-pressure steam supplied to the steam system 70 is supplied from the auxiliary boiler 72. The auxiliary boiler 72 can flexibly adjust the amount of high-pressure steam generated depending on the amount of fuel combusted. Therefore, the hydrocarbon production system 1 using the auxiliary boiler 72 can be said to be a plant having high affinity with the solar steam system 40.

In the steam system 70, the high-pressure steam supplied from the heat recovery device 74, the auxiliary boiler 72, and the solar steam system 40 is converted into power. The converted power is supplied to the utility devices 90 by the power supply line L24. In addition, the steam system 70 can supply heat required in the hydrocarbon production system 1.

Many of the utility devices 90 are supplied with power or heat from the steam system 70. Therefore, the supply amount of the utility (power, electric power, or heat) from the outside can be minimized. As a result, the emission amount of carbon dioxide emitted in a case where the utility is generated from the outside can be reduced. For example, in a case where the electric power generated by using fossil fuel is received from the outside and is used by being converted into power in the plant, carbon dioxide is emitted outside. On the other hand, in the hydrocarbon production system 1, since power is generated in the plant, the emission of carbon dioxide emission occurring outside can be suppressed.

### Second Embodiment

### <<Hydrocarbon Production System>>

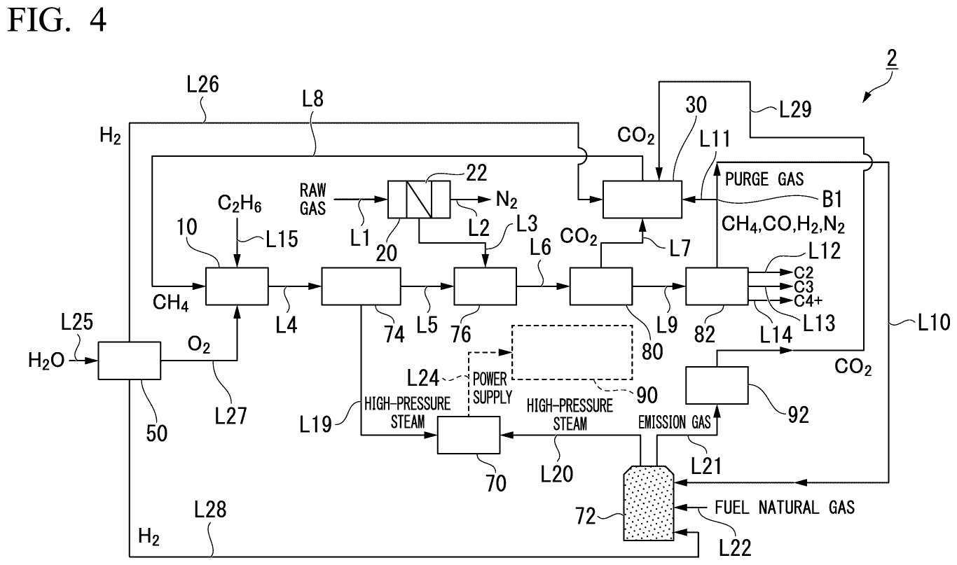

Next, a hydrocarbon production system according to a second embodiment of the present disclosure will be described with reference to FIG. 4. In the following, the same components as those in the first embodiment described above are denoted by the same reference numerals, and the description thereof will be omitted.

As shown in FIG. 4, a hydrocarbon production system 2 according to the present embodiment includes the oxidative coupling reaction device 10, the raw gas separation device 20, the methanation device 30, a water electrolysis device 50, the carbon dioxide separation device 80, and a carbon dioxide recovery device 92.

The water electrolysis device 50 is connected to the methanation device 30 by a pipe L26. The water electrolysis device 50 is connected to the oxidative coupling reaction device 10 by a pipe L27. The water electrolysis device 50 is connected to the auxiliary boiler 72 by a pipe L28. The water electrolysis device 50 is connected to a pipe L25.

The carbon dioxide recovery device 92 is connected to the auxiliary boiler 72 by a pipe L21. The carbon dioxide recovery device 92 and the methanation device 30 are connected to each other by a pipe L29.

### <Water Electrolysis Device>

The water electrolysis device 50 is a device that generates oxygen and hydrogen from water by a water electrolysis reaction (electrolysis of water).

Examples of the water electrolysis device 50 include a device equipped with a water tank, an electrode, and a power source.

### <Carbon Dioxide Recovery Device>

The carbon dioxide recovery device 92 is a device that recovers carbon dioxide in the emission gas.

Examples of the carbon dioxide recovery device 92 include a container filled with an adsorbent capable of selectively adsorbing carbon dioxide and an absorption tower using a carbon dioxide absorbing liquid.

The pipes L25 to L29 are the same as the pipes L1 to L23.

### <<Hydrocarbon Production Method>>

Hereinafter, a hydrocarbon production method of the present embodiment will be described with reference to the hydrocarbon production system 2 of FIG. 4.

The hydrocarbon production method according to the present embodiment has a step of generating oxygen and hydrogen from water by a water electrolysis reaction of the following Formula (9).

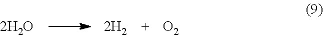

The water supplied from the pipe L25 is applied with a voltage in the water electrolysis device 50 and is decomposed into oxygen and hydrogen. As the electric power of the water electrolysis device 50, for example, a hydrocarbon production system in which the emission amount of carbon dioxide is reduced by using power generation by a solar panel can be adopted. Since the power generation amount of the solar panel varies depending on the sunshine duration and the weather, the electric power supplied to the water electrolysis device 50 can be leveled out by providing an energy storage facility such as a storage battery.

The generated oxygen flows through the pipe L27 and is supplied to the oxidative coupling reaction device 10. In a case where a cryogenic separation device (not shown) and an oxygen separation device (not shown) are used in combination, the amount of oxygen supplied from the oxygen separation device can be reduced.

The generated hydrogen flows through the pipe L26 and is supplied to the methanation device 30. In addition, a part of the generated hydrogen flows through the pipe L28 and is supplied to the auxiliary boiler 72.

The emission gas emitted from the auxiliary boiler 72 flows through the pipe L21 and is supplied to the carbon dioxide recovery device 92.

In the carbon dioxide recovery device 92, carbon dioxide in the emission gas is recovered. The recovered carbon dioxide flows through the pipe L29 and is supplied to the methanation device 30.

In the hydrocarbon production method of the present embodiment, the amount of carbon dioxide that can be converted in the methanation device 30 was simulated by using hydrogen generated by the water electrolysis reaction.

In the simulation, the amount of raw natural gas and the reaction rate (methane conversion rate, C2+selectivity) of the oxidative coupling reaction were assumed by heat and mass balance analysis, and then the amount of steam generated by the waste heat boiler and the amount of CO₂ emitted from a CO₂ separation device were calculated based on the required oxygen supply amount and the amount of reaction heat recovered. In addition, the power required for each of the utility devices 90 was calculated, and the amount of high-pressure steam to be supplied to the steam system 70 was calculated. The amount of steam generated by the auxiliary boiler 72 was calculated by subtracting the amount of steam generated by the waste heat boiler from the amount of high-pressure steam to be supplied to the steam system 70. The amount of the purge gas and the amount of the fuel natural gas satisfying the amount of steam generated from the auxiliary boiler 72 were calculated, and the emission amount of CO₂ emitted from the auxiliary boiler 72 was calculated. In a case where oxygen supplied to the oxidative coupling reaction is produced by an electrolysis reaction of water, the amount of hydrogen generated as a by-product is calculated by the stoichiometric ratio and this hydrogen is supplied as a raw material to the methanation. In this case, the amount of CO₂ in a case where CO₂ is consumed as a raw material by the stoichiometric ratio in the methanation reaction is calculated as the amount of CO₂ that can be reduced. Table 2 shows an example of the results.

TABLE 2

| | | |
|---|---|---|
| Amount of natural gas serving as raw material for | Nm³/h | 268905 |
| reaction | | |
| Amount of purge gas supplied to auxiliary boiler | Nm³/h | 15104 |
| Amount of fuel natural gas supplied to auxiliary | Nm³/h | 22844 |
| boiler | | |
| Amount of oxygen supplied to oxidative coupling | Nm³/h | 216312 |
| reactor | | |
| C2 + production amount | kg/h | 122880 |
| Methane conversion rate | % | 19.4 |
| C2 + selectivity (coupling selectivity) | % | 69.8 |
| Amount of steam generated by waste heat boiler | ton/h | 1085.9 |
| Amount of steam generated by auxiliary boiler | ton/h | 401.7 |
| Emission amount of CO₂ from auxiliary boiler | kg/h | 71507 |
| Emission Amount of CO₂ from CO₂ separation | kg/h | 128367 |
| device | | |
| Total emission amount of CO₂ | kg/h | 199875 |
| Amount of hydrogen supplied to methanation | Nm³/h | 432623 |
| reactor | | |
| Amount of CO₂ that can be reduced by | Nm³/h | 108156 |
| methanation | kg/h | 212449 |

As shown in Table 2, it has been confirmed that the amount of carbon dioxide emitted by the hydrocarbon production method in the related art is 199875 kg/h, whereas 212449 kg/h of carbon dioxide can be reduced by supplying hydrogen generated by the water electrolysis reaction to the methanation and using the carbon dioxide emitted in the related art as a raw material for the methanation. This indicates that the entire amount of carbon dioxide emitted from the auxiliary boiler 72 and the carbon dioxide separation device 80 can be used in the methanation reaction by using the hydrogen generated by the water electrolysis reaction. The surplus hydrogen in the methanation reaction can be used as fuel for the auxiliary boiler 72 as a substitute for the fuel natural gas or the like.

According to the hydrocarbon production method of the present embodiment, since oxygen generated by the water electrolysis reaction is supplied to the oxidative coupling reaction device 10, it is possible to prevent an inert component such as nitrogen from being mixed into oxygen. In a case where oxygen generated by the water electrolysis reaction is used, the supply amount of oxygen into which nitrogen supplied from the cryogenic separation device is mixed can be reduced. Therefore, the amount of nitrogen in the separated gas and the purge gas emitted from the subsequent hydrocarbon separation device 82 can be reduced. As a result, the amount of purge gas combusted in the subsequent auxiliary boiler 72 can be reduced, and the amount of emission gas derived from the combustion of the purge gas fuel emitted from the auxiliary boiler 72 can be reduced. Therefore, the emission amount of carbon dioxide contained in the emission gas can be reduced.

According to the hydrocarbon production method of the present embodiment, since the hydrogen generated in the water electrolysis device 50 is supplied to the methanation device 30, the surplus carbon dioxide that has, in the related art, been recovered in the carbon dioxide separation device 80 and then emitted to the outside (atmosphere) can be used as a raw material for the methanation device 30. Furthermore, since the carbon dioxide in the emission gas of the auxiliary boiler 72 can be effectively utilized as a raw material in the methanation device 30 after being recovered by the carbon dioxide recovery device 92, the emission amount of carbon dioxide emitted to the outside can be reduced.

According to the hydrocarbon production method of the present embodiment, the hydrogen generated in the water electrolysis device 50 is supplied to the auxiliary boiler 72. Therefore, by combusting the hydrogen as fuel, the amount of high-pressure steam can be compensated for by the reduction in the amount of the purge gas. Since the combustion of hydrogen does not generate carbon dioxide, the emission amount of carbon dioxide contained in the emission gas can be reduced. Furthermore, sufficient power can be supplied from the steam system 70 to the utility devices 90, and the emission amount of carbon dioxide emitted in a case where power is generated from the outside.

### Third Embodiment

### <<Hydrocarbon Production System>>

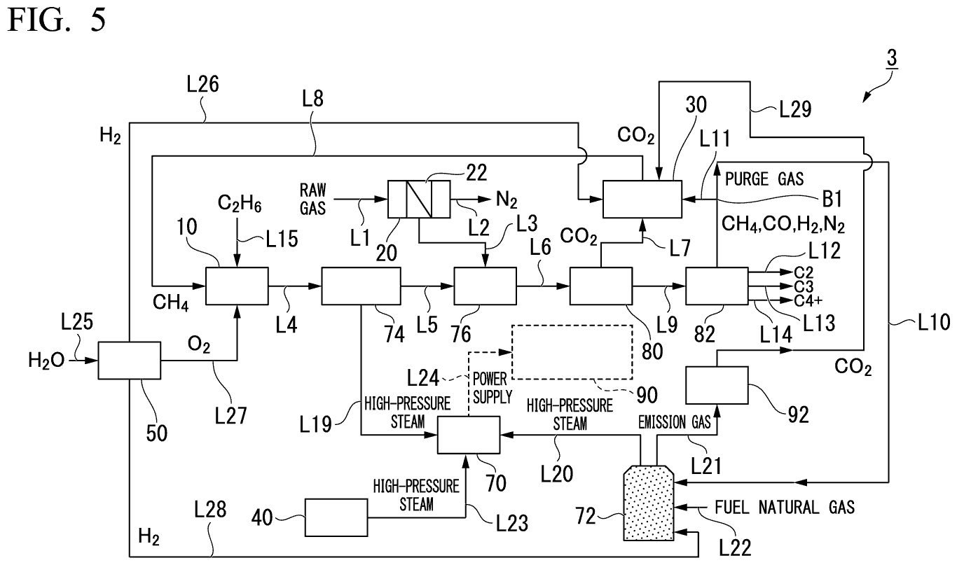

Next, a hydrocarbon production system according to a third embodiment of the present disclosure will be described with reference to FIG. 5. In the following, the same components as those in the first embodiment or the second embodiment described above are denoted by the same reference numerals, and the description thereof will be omitted.

As shown in FIG. 5, a hydrocarbon production system 3 according to the present embodiment includes the oxidative coupling reaction device 10, the raw gas separation device 20, the methanation device 30, the solar steam system 40, the water electrolysis device 50, the carbon dioxide separation device 80, and the carbon dioxide recovery device 92.

The hydrocarbon production system 3 of the present embodiment includes the solar steam system 40 in the hydrocarbon production system 1 of the first embodiment in addition to the hydrocarbon production system 2 of the second embodiment.

The hydrocarbon production system 3 according to the present embodiment can reduce the emission amount of carbon dioxide by reducing the purge gas and the fuel natural gas used as the fuel for the auxiliary boiler 72 by using the solar steam system 40.

### <<Hydrocarbon Production Method>>

The hydrocarbon production method according to the present embodiment is the same as the hydrocarbon production method according to the second embodiment, except that the solar steam system 40 is provided.

Although the embodiments of the present disclosure have been described in detail above, each configuration in each embodiment and a combination thereof are merely examples, and additions, omissions, substitutions, and other modifications of the configuration can be made without departing from the spirit of the present disclosure. In addition, the present disclosure is not limited to the embodiments, and is limited only by the scope of the claims.

For example, the hydrocarbon production system of the present disclosure may include a solar steam system instead of the raw gas separation device.

For example, the hydrocarbon production system of the present disclosure may include a water electrolysis device instead of the raw gas separation device.

In another embodiment, the hydrogen generated by the water electrolysis device and the carbon dioxide recovered by the carbon dioxide recovery device may be supplied to a separate methanation device different from the methanation device, and after methane is generated, the methane may be joined to the gas after the reaction in the methanation device. As a result, since there is no joining of the hydrogen generated by the water electrolysis device and the carbon dioxide recovered by the carbon dioxide recovery device to a recycling line, the concentration variation of the raw gas of the methanation device is eliminated, and complicated operation control is not required.

### <Supplementary Note>

The hydrocarbon production system and hydrocarbon production method described in the above-described embodiments are understood as follows, for example.

(1) Hydrocarbon production systems 1 to 3 according to a first aspect are hydrocarbon production systems that generate a hydrocarbon having two or more carbon atoms from methane by an oxidative coupling reaction of methane, the hydrocarbon production systems including: an oxidative coupling reaction device 10 configured to perform an oxidative coupling reaction between methane and oxygen; a raw gas separation device 20 configured to separate an inert component from a raw gas containing the inert component; a carbon dioxide separation device 80 configured to separate carbon dioxide contained in a generated gas generated in the oxidative coupling reaction device 10; and a methanation device 30 configured to perform a methanation reaction between hydrogen and carbon dioxide.

According to the above-described configuration, since the inert component such as nitrogen is removed from the raw gas, the amount of the inert component in the purge gas emitted from the subsequent hydrocarbon separation device 82 can be reduced. Therefore, the amount of purge gas combusted in the subsequent auxiliary boiler 72 can be reduced, and the amount of emission gas derived from the combustion of the purge gas emitted from the auxiliary boiler 72 can be reduced. As a result, the emission amount of carbon dioxide due to the combustion of the purge gas contained in the emission gas can be reduced.

(2) The hydrocarbon production systems 1 to 3 according to a second aspect are the hydrocarbon production systems 1 to 3 according to (1), in which the raw gas separation device 20 has an inert component separation membrane 22 configured to separate the inert component from the raw gas.

According to the above-described configuration, the inert component can be more reliably separated from the raw gas.

(3) The hydrocarbon production systems 1 to 3 according to a third aspect are the hydrocarbon production systems 1 to 3 according to (1) or (2), in which the methanation device further includes carbon monoxide as a raw material for the methanation reaction.

According to the above-described configuration, carbon monoxide generated in the system can be effectively utilized as a raw material for the methanation reaction.

(4) The hydrocarbon production systems 1 and 3 according to a fourth aspect are the hydrocarbon production systems 1 and 3 according to (1) or (2), further including a solar steam system 40 configured to generate high-pressure steam by using thermal energy from sunlight.

According to the above configuration, since high-pressure steam can be generated without emitting carbon dioxide, the emission amount of carbon dioxide can be reduced.

(5) The hydrocarbon production systems 2 and 3 according to a fifth aspect are the hydrocarbon production systems 2 and 3 according to (1) or (2), further including: a water electrolysis device 50 configured to generate oxygen and hydrogen from water by a water electrolysis reaction.

According to the above-described configuration, since oxygen to be used for the oxidative coupling reaction can be obtained without emitting carbon dioxide, the emission amount of carbon dioxide can be reduced.

(6) The hydrocarbon production systems 2 and 3 according to a sixth aspect are the hydrocarbon production systems 2 and 3 according to (5), in which the oxygen generated in the water electrolysis device 50 is supplied to the oxidative coupling reaction device 10.

According to the above-described configuration, since the oxygen generated by the water electrolysis reaction is supplied to the oxidative coupling reaction device 10, it is possible to prevent the mixing of the inert component that is an impurity. In this case, since the supply amount of the oxygen supplied from the cryogenic separation device 60, in which the inert component is mixed, can be reduced, the amount of the separated gas and the amount of the purge gas emitted from the subsequent hydrocarbon separation device 82 can be reduced. As a result, the amount of purge gas combusted in the subsequent auxiliary boiler 72 can be reduced, and the amount of emission gas derived from the combustion of the purge gas emitted from the auxiliary boiler 72 can be reduced. Therefore, the emission amount of carbon dioxide due to the combustion of the purge gas contained in the emission gas can be reduced.

(7) The hydrocarbon production system 1 according to a seventh aspect is the hydrocarbon production system 1 according to any one of (1) to (6), further including a cryogenic separation device 60 configured to separate oxygen from air; and an oxygen separation device 62, in which the oxygen separation device 62 has an oxygen separation membrane 64 configured to further separate high-purity oxygen from the oxygen separated by the cryogenic separation device 60.

According to the above-described configuration, by removing the inert component from air with the cryogenic separation device 60, the concentration of the inert component in the high-purity oxygen gas supplied to the oxidative coupling reaction device 10 can be reduced. Therefore, the amount of separated gas and purge gas emitted from the subsequent hydrocarbon separation device 82 can be reduced. As a result, the amount of purge gas combusted in the subsequent auxiliary boiler 72 can be reduced, and the amount of emission gas derived from the combustion of the purge gas emitted from the auxiliary boiler 72 can be reduced. Therefore, the emission amount of carbon dioxide due to the combustion of the purge gas contained in the emission gas can be reduced.

(8) The hydrocarbon production systems 2 and 3 according to an eighth aspect are the hydrocarbon production systems 2 and 3 according to (5) or (6), in which the hydrogen generated in the water electrolysis device 50 is supplied to the methanation device 30.

According to the above-described configuration, the raw natural gas can be reduced by the amount of methane generated by the methanation, and the economy of a plant can be further improved.

(9) The hydrocarbon production systems 2 and 3 according to a ninth aspect are the hydrocarbon production systems 2 and 3 according to any one of (1) to (8), further including: a carbon dioxide recovery device 92, in which recovered carbon dioxide is supplied to the methanation device 30.

According to the above-described configuration, since carbon dioxide in the emission gas can be effectively utilized by the methanation device 30, the emission amount of carbon dioxide emitted to the outside can be reduced.

(10) The hydrocarbon production systems 1 and 3 according to a tenth aspect are hydrocarbon production systems that generate a hydrocarbon having two or more carbon atoms from methane by an oxidative coupling reaction of methane, the hydrocarbon production systems including: an oxidative coupling reaction device 10 configured to perform an oxidative coupling reaction between methane and oxygen; a carbon dioxide separation device 80 to separate carbon dioxide contained in a generated gas generated by the oxidative coupling reaction device 10; a methanation device 30 configured to perform a methanation reaction between hydrogen and carbon dioxide; and a solar steam system 40 configured to generate high-pressure steam by using thermal energy from sunlight.

According to the above-described configuration, since high-pressure steam can be generated using solar heat as energy without emitting carbon dioxide, the emission amount of carbon dioxide can be reduced.

(11) The hydrocarbon production systems 2 and 3 according to an eleventh aspect are hydrocarbon production systems that generate a hydrocarbon having two or more carbon atoms from methane by an oxidative coupling reaction of methane, the hydrocarbon production systems including: an oxidative coupling reaction device 10 configured to perform an oxidative coupling reaction between methane and oxygen; a carbon dioxide separation device 80 configured to separate carbon dioxide contained in a generated gas generated by the oxidative coupling reaction device 10; a methanation device 30 configured to perform a methanation reaction between hydrogen and carbon dioxide; and a water electrolysis device 50 configured to generate oxygen and hydrogen from water by a water electrolysis reaction.

According to the above-described configuration, oxygen to be used for the oxidative coupling reaction can be obtained with high purity. Therefore, by reducing the oxygen supply amount from the cryogenic separation device, the amount of carbon dioxide in the emission gas derived from the combustion of the purge gas can be reduced, and the emission amount of carbon dioxide can be reduced.

(12) A hydrocarbon production method according to a twelfth aspect is a hydrocarbon production method for generating a hydrocarbon having two or more carbon atoms from methane by an oxidative coupling reaction of methane, the method including a step S1 of generating a hydrocarbon having two or more carbon atoms from methane by the oxidative coupling reaction; a step S2 of separating an inert component from a raw gas containing the inert component; a step S3 of separating carbon dioxide included in a generated gas generated by the oxidative coupling reaction; and a step S4 of generating methane from hydrogen and carbon dioxide by a methanation reaction.

According to the above-described configuration, since the inert component is removed from the raw gas, the amount of purge gas emitted in the subsequent hydrocarbon separation step S6 can be reduced. Therefore, the amount of purge gas to be combusted in the subsequent combustion step S7 can be reduced, and the amount of emission gas derived from the combustion of the purge gas emitted in the combustion step S7 can be reduced. As a result, the emission amount of carbon dioxide due to the combustion of the purge gas contained in the emission gas can be reduced.

(13) The hydrocarbon production method according to a thirteenth aspect is the hydrocarbon production method according to (12), in which the inert component is separated from the raw gas by an inert component separation membrane.

According to the above-described configuration, the inert component can be more reliably removed from the raw gas.

(14) The hydrocarbon production method according to a fourteenth aspect is the hydrocarbon production method according to (12) or (13), further including: carbon monoxide as a raw material for the methanation reaction.

According to the above-described configuration, carbon monoxide generated in the system can be effectively utilized as a raw material for the methanation reaction.

(15) The hydrocarbon production method according to a fifteenth aspect is the hydrocarbon production method according to any one of (12) to (14), further including: a step S8 of generating high-pressure steam by using thermal energy from sunlight.

According to the above configuration, since high-pressure steam can be generated without emitting carbon dioxide, the emission amount of carbon dioxide can be reduced.

(16) The hydrocarbon production method according to a sixteenth aspect is the hydrocarbon production method according to any one of (12) to (15), further including: a step of generating oxygen and hydrogen from water by a water electrolysis reaction.

According to the above-described configuration, since oxygen to be used for the oxidative coupling reaction can be obtained without emitting carbon dioxide, the emission amount of carbon dioxide can be reduced. In addition, since the oxygen produced in the water electrolysis reaction having no mixing of the inert component such as nitrogen into oxygen, the amount of purge gas can be reduced, and the amount of emission gas derived from the combustion of the purge gas emitted from the auxiliary boiler 72 can be reduced.

(17) The hydrocarbon production method according to a seventeenth aspect is the hydrocarbon production method according to (16), in which oxygen generated by the water electrolysis reaction is supplied to the oxidative coupling reaction.

According to the above-described configuration, since the oxygen generated by the water electrolysis reaction is supplied to the oxidative coupling reaction, it is possible to prevent the mixing of the inert component that is an impurity. In this case, since the supply amount of oxygen into which the inert component is mixed can be reduced, the amount of purge gas emitted in the subsequent hydrocarbon separation step S6 can be reduced. Therefore, the amount of purge gas to be combusted in the subsequent combustion step S7 can be reduced, and the amount of emission gas derived from the combustion of the purge gas emitted in the combustion step S7 can be reduced. As a result, the emission amount of carbon dioxide due to the combustion of the purge gas contained in the emission gas can be reduced.

(18) The hydrocarbon production method according to an eighteenth aspect is the hydrocarbon production method according to any one of (12) to (17), further including: a step S5-1 and a step S5-2 of separating oxygen from air.

According to the above-described configuration, by removing the inert component from air, the concentration of the inert component in the high-purity oxygen gas supplied to the oxidative coupling reaction can be reduced. Therefore, the amount of separated gas and purge gas emitted in the subsequent hydrocarbon separation step S6 can be reduced. As a result, the amount of purge gas to be combusted in the subsequent combustion step S7 can be reduced, and the amount of emission gas derived from the combustion of the purge gas emitted in the combustion step S7 can be reduced. Therefore, the emission amount of carbon dioxide due to the combustion of the purge gas contained in the emission gas can be reduced.

(19) The hydrocarbon production method according to a nineteenth aspect is the hydrocarbon production method according to any one of (16) to (18), in which the hydrogen generated by the water electrolysis reaction is supplied to the methanation reaction.

According to the above-described configuration, the emission amount of carbon dioxide can be reduced by effectively utilizing carbon dioxide emitted into the atmosphere as a raw material for methanation. In addition, the amount of synthetic methane generated can be further increased. Therefore, the consumption amount of the raw gas can be suppressed, which is economical.

(20) The hydrocarbon production method according to a twentieth aspect is the hydrocarbon production method according to any one of (12) to (19), further including a step of recovering carbon dioxide, in which the recovered carbon dioxide is supplied to the methanation reaction.

According to the above-described configuration, since carbon dioxide in the emission gas can be effectively utilized in the methanation reaction without being emitted into the atmosphere, the emission amount of carbon dioxide emitted to the outside can be reduced.

(21) Hydrocarbon production systems 1 to 3 according to a twenty-first aspect are hydrocarbon production systems that generate a hydrocarbon having two or more carbon atoms from methane by an oxidative coupling reaction of methane, the hydrocarbon production systems including: an oxidative coupling reaction device 10 configured to perform an oxidative coupling reaction between methane and oxygen; a raw gas separation device 20 to separate an inert component from a raw gas containing the inert component; a methanation device 30 configured to perform a methanation reaction between hydrogen and carbon dioxide; a carbon dioxide separation device 80 configured to separate carbon dioxide contained in a generated gas generated by the oxidative coupling reaction device 10; and a hydrocarbon separation device 82 configured to separate a targeted hydrocarbon from a gas containing the hydrocarbon, in which the raw gas separation device 20 has an inert component separation membrane 22 configured to separate the inert component from the raw gas, the raw gas from which at least a part of the inert component is separated is supplied to the oxidative coupling reaction device 10 through any gas treatment device, a recycling gas containing methane is supplied from the methanation device 30 to the oxidative coupling reaction device 10, the oxidative coupling reaction device 10 generates a generated gas containing the hydrocarbon by an oxidative coupling reaction between the oxygen and methane contained in the recycling gas, the generated gas is supplied from the oxidative coupling reaction device 10 to the hydrocarbon separation device 82 through any gas treatment device, the hydrocarbon separation device 82 separates a targeted hydrocarbon from the generated gas to generate a separated gas containing at least hydrogen, the separated gas is supplied from the hydrocarbon separation device 82 to the methanation device 30, the methanation device 30 generates methane from hydrogen contained in the separated gas and carbon dioxide supplied from any carbon dioxide supply source, and the separated gas is supplied as necessary to an auxiliary boiler 72 as a purge gas.

According to the above-described configuration, since the inert component such as nitrogen is removed from the raw gas, the amount of the inert component such as the purge gas emitted from the hydrocarbon separation device 82 can be reduced. Therefore, the amount of purge gas combusted in the subsequent auxiliary boiler 72 can be reduced, and the amount of emission gas derived from the combustion of the purge gas emitted from the auxiliary boiler 72 can be reduced. As a result, the emission amount of carbon dioxide due to the combustion of the purge gas contained in the emission gas can be reduced.

### INDUSTRIAL APPLICABILITY

According to the hydrocarbon production system and the hydrocarbon production method of the present disclosure, the emission amount of carbon dioxide can be reduced in a case where a hydrocarbon is produced by the OCM reaction.

### REFERENCE SIGNS LIST

1, 2, 3 Hydrocarbon production system
10 Oxidative coupling reaction device
20 Raw gas separation device
22 Inert component separation membrane
30 Methanation device
40 Solar steam system
50 Water electrolysis device
60 Cryogenic separation device
62 Oxygen separation device
64 Oxygen separation membrane
70 Steam system
72 Auxiliary boiler
74 Heat recovery device
76 Booster device
80 Carbon dioxide separation device
82 Hydrocarbon separation device
90 Utility device
92 Carbon dioxide recovery device

1. A hydrocarbon production system that generates a hydrocarbon having two or more carbon atoms from methane by an oxidative coupling reaction of methane, the hydrocarbon production system comprising:
an oxidative coupling reaction device configured to perform an oxidative coupling reaction between methane and oxygen;
a raw gas separation device configured to separate an inert component from a raw gas containing the inert component;
a carbon dioxide separation device configured to separate carbon dioxide contained in a generated gas generated in the oxidative coupling reaction device; and
a methanation device configured to perform a methanation reaction between hydrogen and carbon dioxide, and
a cryogenic separation device configured to separate oxygen from air; and
an oxygen separation device,
wherein the oxygen separation device has an oxygen separation membrane configured to further separate high-purity oxygen from the oxygen containing nitrogen as a main component of an inert gas, which is separated by the cryogenic separation device.

2. The hydrocarbon production system according to claim 1,
wherein the raw gas separation device has an inert component separation membrane configured to separate the inert component from the raw gas.

3. The hydrocarbon production system according to claim 1,
wherein the methanation device further includes carbon monoxide as a raw material for the methanation reaction.

4. The hydrocarbon production system according to claim 1, further comprising:
a solar steam system configured to generate high-pressure steam by using thermal energy from sunlight.

5. The hydrocarbon production system according to claim 1, further comprising:
a water electrolysis device configured to generate oxygen and hydrogen from water by a water electrolysis reaction.

6. The hydrocarbon production system according to claim 5,
wherein oxygen generated in the water electrolysis device is supplied to the oxidative coupling reaction device.

7. (canceled)

8. The hydrocarbon production system according to claim 5,
wherein hydrogen generated in the water electrolysis device is supplied to the methanation device.

9. The hydrocarbon production system according to claim 1, further comprising:
a carbon dioxide recovery device,
wherein recovered carbon dioxide is supplied to the methanation device.

10. A hydrocarbon production system that generates a hydrocarbon having two or more carbon atoms from methane by an oxidative coupling reaction of methane,
the hydrocarbon production system comprising:
an oxidative coupling reaction device configured to perform an oxidative coupling reaction between methane and oxygen;
a carbon dioxide separation device configured to separate carbon dioxide contained in a generated gas generated in the oxidative coupling reaction device;
a methanation device configured to perform a methanation reaction between hydrogen and carbon dioxide; and
a solar steam system configured to generate high-pressure steam by using thermal energy from sunlight.

11. A hydrocarbon production system that generates a hydrocarbon having two or more carbon atoms from methane by an oxidative coupling reaction of methane, the hydrocarbon production system comprising:
an oxidative coupling reaction device configured to perform an oxidative coupling reaction between methane and oxygen;
a carbon dioxide separation device configured to separate carbon dioxide contained in a generated gas generated by the oxidative coupling reaction device;
a methanation device configured to perform a methanation reaction between hydrogen and carbon dioxide; and
a water electrolysis device configured to generate oxygen and hydrogen from water by a water electrolysis reaction, and
a cryogenic separation device configured to separate oxygen from air; and
an oxygen separation device,
wherein the oxygen separation device has an oxygen separation membrane configured to further separate high-purity oxygen from the oxygen containing nitrogen as a main component of an inert gas, which is separated by the cryogenic separation device.

12. A hydrocarbon production method for generating a hydrocarbon having two or more carbon atoms from methane by an oxidative coupling reaction of methane, the hydrocarbon production method comprising:
a step of generating a hydrocarbon having two or more carbon atoms by an oxidative coupling reaction of methane;
a step of separating an inert component from a raw gas containing the inert component;
a step of separating carbon dioxide contained in a generated gas generated by the oxidative coupling reaction; and
a step of generating methane from hydrogen and carbon dioxide by a methanation reaction, and
a cryogenic separation step of separating oxygen from air; and
an oxygen separation step of further separating high-purity oxygen by using an oxygen separation membrane from the oxygen containing nitrogen as a main component of an inert gas, which is separated by the cryogenic separation step.

13. The hydrocarbon production method according to claim 12,
wherein the inert component is separated from the raw gas by an inert component separation membrane.

14. The hydrocarbon production method according to claim 12,
wherein carbon monoxide is further contained as a raw material for the methanation reaction.

15. The hydrocarbon production method according to claim 12, further comprising:
a step of generating high-pressure steam by using thermal energy from sunlight.

16. The hydrocarbon production method according to claim 12, further comprising:
a step of generating oxygen and hydrogen from water by a water electrolysis reaction.

17. The hydrocarbon production method according to claim 16,
wherein oxygen generated by the water electrolysis reaction is supplied to the oxidative coupling reaction.

18. (canceled)

19. The hydrocarbon production method according to claim 16,
wherein hydrogen generated by the water electrolysis reaction is supplied to the methanation reaction.

20. The hydrocarbon production method according to claim 19, further comprising:
a step of recovering carbon dioxide,
in which the recovered carbon dioxide is supplied to the methanation reaction.
